(19) United States
(12) Patent Application Publication
(10) Pub. No.: US 2014/0106373 A1
(43) Pub. Date: Apr. 17, 2014

(54) METHOD FOR TARGET PROTEIN NORMALIZATION

(71) Applicant: GE HEALTHCARE BIO-SCIENCES AB, UPPSALA (SE)

(72) Inventors: Asa Hagner-Mcwhirter, UPPSALA (SE); Ola Ronn, UPPSALA (SE)

(73) Assignee: GE HEALTHCARE BIO-SCIENCES AB, UPPSALA (SE)

(21) Appl. No.: 14/122,382

(22) PCT Filed: May 29, 2012
(86) PCT No.: PCT/SE12/50571

§ 371 Date: Nov. 26, 2013

(30) Foreign Application Priority Data
May 31, 2011 (SE) 1150503-9

(51) Int. Cl. G01N 33/566

(52) U.S. Cl. CPC G01N 33/566
USPC 435/7.9; 436/501

(57) ABSTRACT

The present invention relates to a method for target protein normalization, especially for Western blotting applications. More closely, the invention relates to a method for normalizing target protein signals, after electrophoresis and Western blotting, against variations of sample load or cell number between different lanes or within the same lane on an electrophoretic gel. The signals are normalized against the total protein signal (=ratio between target protein/total protein) or reference protein band signal(s) (=ratio between target protein/reference protein band).

According to the invention multiplex and quantitative assessments are possible, such as quantitative comparison between target proteins in different samples.

### FIELD OF THE INVENTION

The present invention relates to a method for target protein normalization, especially for Western blotting applications. More closely, the invention relates to a method for normalizing target protein signals, after electrophoresis and Western blotting, against variations of sample load or cell number between different lanes or within the same lane on an electrophoretic gel. According to the invention multiplex and quantitative assessments are possible, such as quantitative comparison between target proteins in different samples.

### BACKGROUND OF THE INVENTION

Western blotting (or, protein immunoblotting) is an analytical technique used to detect specific proteins in a given sample of tissue homogenate, cell lysate or other protein containing samples. It uses gel electrophoresis to separate native or denatured proteins by the length of the polypeptide (denaturing conditions) or by the 3-D structure of the protein (native/non-denaturing conditions). The proteins are then transferred to a membrane (typically nitrocellulose or PVDF), where they are probed (detected) using antibodies specific to the target protein. For certain applications, the proteins are probed in the gel without a transfer step. However, further description of Western blot methodology is based on protein samples transferred to a membrane.

During the detection process the membrane is probed for the protein of interest by the use of an antibody specific for the protein of interest. Due to possibilities of increased signal amplification and to avoid negative effects on target specific affinity related to primary antibody conjugation, this traditionally takes place in a two-step process (using a primary target specific antibody and a secondary labeled antibody specific for the primary antibody), although there are now one-step detection methods available for certain applications. The one-step method allows the process to occur faster and with a lower amount of consumables, but sensitivity may be compromised. This requires a probe antibody which both recognizes the protein of interest and contains a detectable label, probes which are often available for known protein tags. The primary probe is incubated with the membrane in a manner similar to that for the primary antibody in a two-step process, and is then ready for direct detection after a series of wash steps.

Today, normalization and loading control is usually done by detection of an endogenously constitutively expressed internal standard or house-keeping protein such as GAPDH, tubulin or actin. These proteins are assumed to be constitutively expressed by the cells, independently of time or treatment, and reflect sample load or number of cells in each lane. However, reliance of house-keeping proteins for normalization does not always lead to accurate results. On the contrary, it has been shown that the expression of house-keeping proteins can be affected by treatments or conditions. In these cases the level of house-keeping protein no longer reflects cell number and cannot be a reliable standard for normalization between samples that is correcting for uneven sample loading. This will lead to inaccurate results.

There is also a general concern that in some samples the slope of curve for protein amount vs. signal intensity is different for target and house-keeping protein, making normalization inaccurate (ratio will vary depending on protein amount). Another drawback with the conventional use of house-keeping proteins is that they require antibody based detection which reduces the number of targets available for multiplexing. Thus it would be desirable not having to spend any antibodies on detection of house-keeping proteins.

All hitherto known methods for quantification of Western blotting applications rely on labeling of the sample target proteins on the membrane in steps following the blotting procedure. Pre-labeling of total sample protein or reference protein(s) has not been suggested.

Thus, it would be desired to provide a more reliable and simple procedure for normalization of sample load in electrophoretic applications, such as Western blotting.

### SUMMARY OF THE INVENTION

The present invention provides a novel normalization method, especially suited for Western blotting applications.

In a first aspect, the invention relates to a method for normalizing target protein signal(s), after electrophoresis, from protein band(s) in a separate lane on an electrophoretic gel against initial sample load or initial cell number loaded onto said gel, wherein the signal is normalized against the total protein signal (=ratio between target protein/total protein) or reference protein band signal(s) (=ratio between target protein/reference protein band) in the same lane as said target protein band(s) for qualitative determination (and optionally comparison) of target protein(s) in the same and in different lanes, and wherein all sample proteins are pre-labeled, i.e. before electrophoresis, with the same dye.

Preferably, the pre-labeling is with a fluorescent dye. Most preferably a fluorescent cyanine dye or any other dye enabling simultaneous detection separately from immune detected signals.

In one embodiment the pre-labeled sample proteins from the gel are blotted over to a membrane and the membrane is probed with primary antibodies, Fab-fragments or other affinity binder against a first target protein in the sample load, and thereafter with secondary antibodies labeled with a second dye for visualizing said first target protein.

Simultaneously or sequentially, the membrane may be probed with further primary antibodies against a second target protein and then with secondary antibodies labeled with a third dye for visualizing said second target protein. Simultaneously or sequentially, the membrane may be probed with further primary antibodies against a third target protein and then secondary antibodies labeled with a fourth dye for visualizing said third target protein.

In an alternative embodiment, the pre-labeled sample proteins from the gel are blotted over to a membrane and the membrane is probed with primary antibodies against one or more target proteins in the sample load and the primary antibodies are differentially labeled to allow multiplexing.

The invention provides a method for quantitative Western blotting comprising the following steps: a) pre-labeling all proteins in one or more samples with a first dye, b) running electrophoresis of said sample proteins, c) blotting over said sample proteins to a Western blotting membrane, d) probing said membrane with one or more primary antibodies against one or more target proteins among said sample proteins wherein the primary antibodies are differentially labeled, and e), determining the quantity of said target protein(s) in relation to said pre-labeled sample protein(s) and optionally in relation to one or more target protein(s) among the sample proteins.

An alternative method for quantitative Western blotting comprises the following steps: a) pre-labeling all proteins in one or more samples with a first dye, b) running electrophoresis of said sample proteins, c) blotting over said sample proteins to a Western blotting membrane, d) probing said membrane with one or more primary antibodies against one or more target proteins among said sample proteins, e) adding one or more secondary antibodies labeled with a second dye and a third dye etc to label ad first and second target protein etc, and f) determining the quantity of said target protein(s) in relation to said pre-labeled sample protein(s) and optionally in relation to one or more target protein(s) among the sample proteins.

The dyes may be selected from Cy 2, Cy 3, Cy 5 and Cy 7, or any other suitable dyes enabling multiplexing.

### BRIEF DESCRIPTION OF THE FIGURES

FIG. 1 shows a schematic overview of the workflow according to the invention of normalizing detected targets on a Western membrane to pre-labeled total proteins.

FIG. 2 shows that the ratio between target and pre-labeled total protein bands is constant independently of sample amount indicating accuracy of normalization method.

### DETAILED DESCRIPTION OF THE INVENTION

The present invention provides a new approach for between lane normalization or loading control especially for fluorescent Western blotting applications. The advantage is to combine pre-labeling fluorescent Western blotting and the fact that the labeled proteins will be transferred onto a membrane (due to covalent bond between dye and protein) enabling normalization of the specific Western signals.

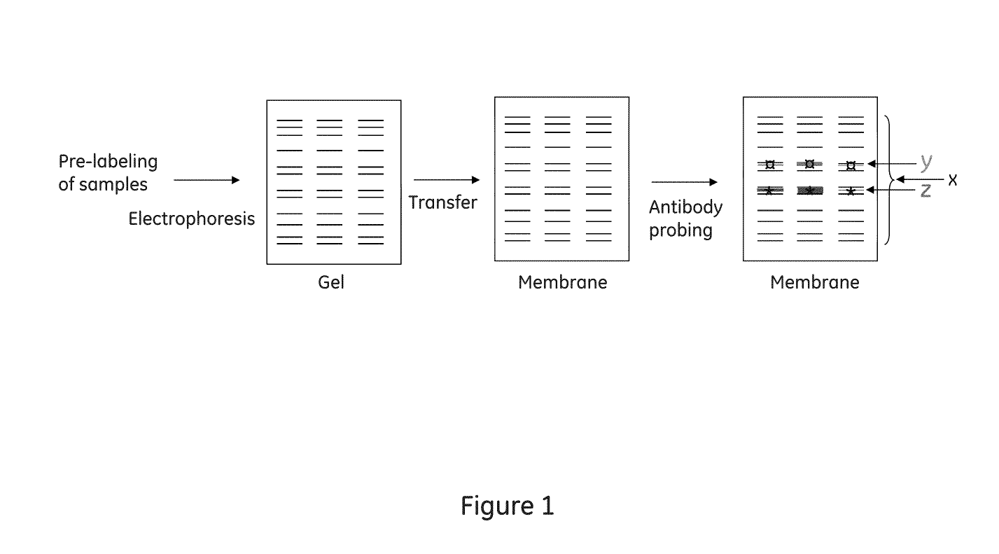

FIG. 1 shows samples covalently pre-labeled with CyDye x (eg. Cy2- NHS reactive group), transferred from a SDS PAGE gel to a Western membrane and finally probed with primary antibodies from different species directed against the target proteins and secondary antibodies (directed against different species of primary antibodies) conjugated with CyDye y and CyDye z (eg. Cy3 and Cy5).

The target proteins z and y within each lane are normalized against the pre-labeled total protein bands or a selection of pre-labeled total protein(s) according to: y/x or z/x Or, the pre-labeled total protein x signal lane 1 is set to 100% and the x signal in lane 2 and 3 etc is related to lane 1 by a factor (lane 1 x/lane 2 x etc) that is then used to adjust/normalize the target protein of interest signals (z and y)

After normalization of the signals the signal intensities between lanes can be directly compared with each other to achieve quantitative and accurate results of target protein levels in the different samples.

CyDye pre-labeled samples are separated by 1-D electrophoresis and the proteins are transferred to the membrane. Both labeled and unlabeled proteins will be transferred to the membrane and the ratio labeled: unlabeled will depend on the type of labeling. For minimal CyDye labeling only 1-3% of the proteins will be labeled and the majority will be unlabeled. For saturation labeling most of the proteins will be labeled. The membrane is then blocked and probed with primary and secondary-CyDye conjugated antibodies for up to three targets in addition to pre-labeled sample (Cy2, Cy3, Cy5 or Cy7 simultaneously multiplexed on the membrane). According to the invention the CyDye labeled total protein signal from all bands within the whole lane is used for normalization, leaving up to three detection channels available for detection of target proteins.

Alternatively, for less complex samples (enriched or fractionated samples), if possible (sufficient resolution and signal strength of total protein bands), the pre-labeled signal from the target protein itself is used for normalization. The CyDye conjugation will add approximately 500 Da to the protein molecular weight. Therefore, migration position in the gel of the pre-labeled band is expected to be above the Western signal (detecting mainly unlabeled proteins), depending on size of target protein (larger shift for smaller proteins).

The new approach described here will be more accurate for all samples and applications since it is the total protein signal within the whole lane that is used for normalization. The total protein from a CyDye pre-labeled sample is proportional to sample amount as long as signals are detected below the upper threshold of detection of the imager used, i.e. no saturated signals are used for the analysis. Saturated signals are no longer proportional to protein amount as detected pixel values are truncated and are avoided by using optimized intensity settings when scanning the membrane according to Imager instructions. However, this method should not be considered as an absolute quantification method for total proteins and only samples from the same source (cells or tissue), pre-labeled with the same dye and detected with the same Imager settings (channel and intensity) can be relatively compared.

This new method for normalization will also enable simultaneous detection of up to three proteins of interests with three primary antibodies compared with the current procedure in which one primary antibody is occupied for detection of a house-keeping protein.

Thus the invention provides an easy and advantageous solution for normalization of for example Western signals for fluorescent 1D SDS PAGE with pre-labeled samples which is expected to be of high and general increasing interest for quantitative protein analysis by Western blotting.

### Experimental Part

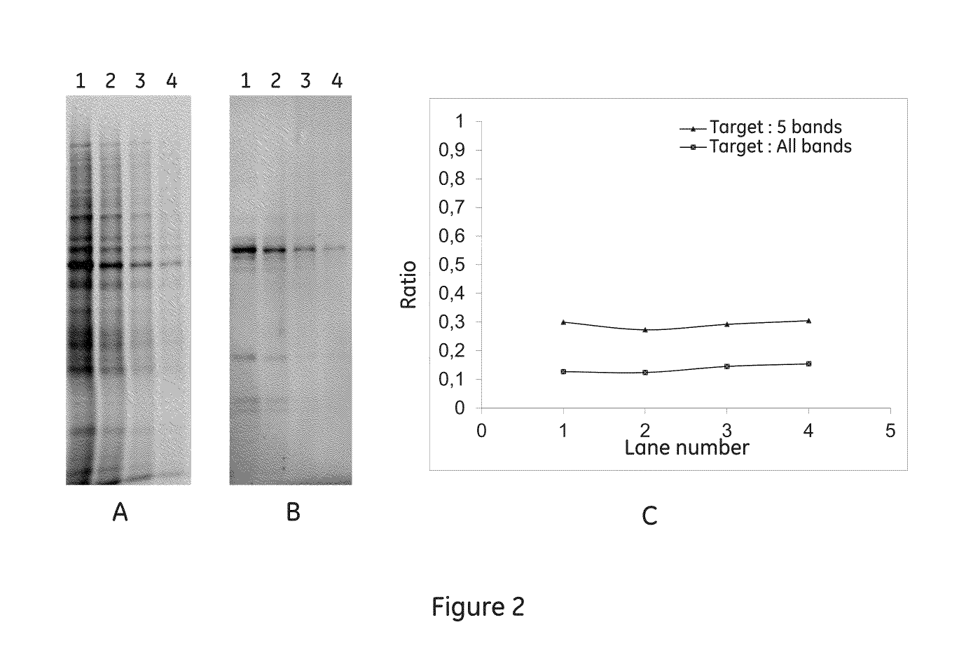

The invention will now be described more closely in association with the accompanying drawing, FIG. 2. HeLa cell lysate in a 2-fold dilution series from 5 μ•••••••••μ••••••••••••••was pre-labeled with Cy5 (NHS reactive group) and subjected to 1D SDS PAGE. The Cy5 pre-labeled proteins in the gel were transferred to a membrane and probed with primary antibody from rabbit against ERK followed by secondary anti-rabbit IgG conjugated with Cy3 (ECL Plex Cy3). The membrane was scanned in Cy5 channel for detection of Cy5 pre-labeled total protein (A) and in Cy3 channel for detection of target protein ERK (B) using a fluorescent Imager. The ratio between either the 5 strongest or all Cy5 pre-labeled total protein bands was used for calculating the ratio of target protein: pre-labeled total protein (C).

A 2-fold dilution series of 5, 2.5, 1.25, and 0.6 μg Hela cell lysate (Santa Cruz Biotechnology) were pre-labeled with Cy™ 5 NHS ester (GE Healthcare) and separated by 1D SDS PAGE (8×7 cm 12.5% Tris-Glycine, 15 well, Novex, Invitrogen). The proteins were transferred onto a low-fluorescent membrane (Hybond LFP™, GE Healthcare). The membrane was blocked using a low-fluorescent blocking agent (2% ECL Advance blocking agent (GE Healthcare) in PBS 0.1% Tween-20) and probed with rabbit anti-ERK primary antibody (Polyclonal anti-mitogen activated kinase (Erk1/Erk2, Sigma) diluted 1:5000, and an ECL Plex™ Cy 3 secondary antibody (anti rabbit IgG CyDye conjugated, GE Healthcare) diluted 1:2500. Multiplex ECL Plex antibody signals and CyDye pre-labeled protein signals were detected separately on the same membrane by using a fluorescent imager and different detection channels (Typhoon™ FLA9000 Imager, GE Healthcare). Image analysis was performed using ImageQuant™TL image analysis software (GE Healthcare).

The method can be summarized with the following example:
1. Pre-labeling of total protein samples with Cy 2, 1-D electrophoresis, transfer, blocking and probing of membrane according to ECL Plex protocol.
2. Image analysis of the membrane using software, Cy 2 total protein signals within each lane is measured and related to the signals of target proteins detected with ECL Plex Cy 5 and/or ECL Plex Cy 3. Or, if sample is fractionated or enriched, ECL Plex Cy 5 and/or ECL Plex Cy 3 target protein signals may be related to Cy 2 protein signal of the corresponding target protein.

1. A method for normalizing target protein signal(s), after electrophoresis, from protein band(s) in a separate lane on an electrophoretic gel against initial sample load or initial cell number loaded onto said gel, wherein the signal is normalized against the total protein signal (=ratio between target protein/total protein) or reference protein band signal(s) (=ratio between target protein/reference protein band) in the same lane as said target protein band(s) for qualitative determination (and optionally comparison) of target protein(s) in the same and in different lanes, and wherein all sample proteins are pre-labeled, i.e. before electrophoresis, with a same first dye.

2. The method of claim 1, wherein the pre-labeling is with a fluorescent dye.

3. The method of claim 2, wherein the pre-labeling is with a fluorescent cyanine dye.

4. The method of claim 1, wherein the pre-labeled sample proteins from the gel are blotted over to a membrane and the membrane is probed with primary antibodies, Fab fragments or other affinity binder against a first target protein in the sample load, and thereafter with secondary antibodies labeled with a second dye for visualizing said first target protein.

5. The method of claim 4, comprising probing the membrane with further primary antibodies against a second target protein and then with secondary antibodies labeled with a third dye for visualizing said second target protein.

6. The method of claim 5, comprising probing the membrane with further primary antibodies against a third target protein and then secondary antibodies labeled with a fourth dye for visualizing said third target protein.

7. The method of claim 4, comprising probing the membrane with further primary antibodies against a second target protein and then with secondary biotin conjugated antibodies followed by Dye or reporter enzyme (HRP, AP) conjugated Streptavidin visualizing target proteins.

8. The method of claim 4, comprising probing the membrane with further primary antibodies against a second target protein and then with secondary antibodies conjugated with horse radish peroxidas (HRP), alkaline phosphatase (AP) or other reporter enzyme for visualizing said target protein after incubation with chemiluminescent (ECL) or colorimetric substrate.

9. The method of claim 1, wherein the pre-labeled sample proteins from the gel are blotted over to a membrane and the membrane is probed with primary antibodies against one or more target proteins in the sample load and the primary antibodies are differentially labeled.

10. The method of claim 1 for quantitative Western blotting comprising the following steps:
a) pre-labeling all proteins in one or more samples with a first dye;
b) running electrophoresis of said sample proteins;
c) blotting over said sample proteins to a Western blotting membrane;
d) probing said membrane with one or more primary antibodies against one or more target proteins among said sample proteins wherein the primary antibodies are differentially labeled; and
e) determining the quantity of said target protein(s) in relation to said pre-labeled sample protein(s) and optionally in relation to one or more target protein(s) among the sample proteins.

11. The method of claim 1 for quantitative Western blotting comprising the following steps:
a) pre-labeling all proteins in one or more samples with a first dye;
b) running electrophoresis of said sample proteins;
c) blotting over said sample proteins to a Western blotting membrane;
d) probing said membrane with one or more primary antibodies against one or more target proteins among said sample proteins;
e) adding one or more secondary antibodies labeled with a second dye and a third dye etc to label first and second target protein etc; and
f) determining the quantity of said target protein(s) in relation to said pre-labeled sample protein(s) and optionally in relation to one or more target protein(s) among the sample proteins.

12. The method of claim 10, wherein the dyes are selected from Cy 2, Cy 3, Cy 5 and Cy 7.

13. The method of claim 11, wherein the dyes are selected from Cy 2, Cy 3, Cy 5 and Cy 7.
